(19) United States
(12) Patent Application Publication
(10) Pub. No.: US 2026/0007378 A1
(43) Pub. Date: Jan. 8, 2026

(54) Lightweight Curved Support and Guide Rail with High Load Capacity for C-Arm Imaging Systems

(71) Applicant: GE Precision Healthcare LLC, Waukesha, WI (US)

(72) Inventors: Julien Marcotte, Saint Vrain (FR); Bernard Bouvier, Eragny (FR)

(21) Appl. No.: 18/761,879

(22) Filed: Jul. 2, 2024

(51) Int. Cl. A61B 6/00

(52) U.S. Cl. CPC A61B 6/4441

(57) ABSTRACT

A C-shaped arm for use with a medical imaging system includes a C-shaped portion, a radiation source carried by the C-shaped portion, a radiation detector carried by the C-shaped portion, and a pair of guide rails secured to opposed sides of the C-shaped portion, wherein each of the pair of guide rails has a body formed of a lightweight material and including a pair of rod channels formed therein and a pair of rods engaged within the pair of rod channels. The body can be formed by extruding the lightweight material into a unitary structure for the body with the pair of rods engaged at least partially within the pair of rod channels using an interference fit either prior to or after bending the body into the desired curved shape for the guide rail.

### FIELD OF THE DISCLOSURE

The subject matter disclosed herein relates to X-ray imaging systems having C-arms and, more particularly, to curved support and guide rails for C-arm that are light weight and have a high load capacity.

### BACKGROUND OF THE DISCLOSURE

Medical diagnostic imaging systems generate images of an object, such as a patient, for example, through exposure to an energy source, such as X-rays passing through a patient, for example. The generated images may be used for many purposes. Often, when a practitioner takes X-rays of a patient, it is desirable to take several X-rays of one or more portions of the patient's body from a number of different positions and angles, and preferably without needing to frequently reposition the patient. To meet this need, C-arm X-ray diagnostic equipment has been developed. The term C-arm generally refers to an X-ray imaging device having a rigid and/or articulating structural member having an X-ray source and an image detector assembly that are each located at an opposing end of the structural member so that the X-ray source and the image detector face each other. The structural member is typically “C” shaped and so is referred to as a C-shaped arm or C-arm. In this manner, X-rays emitted from the X-ray source can impinge on the image detector and provide an X-ray image of the object or objects that are placed between the X-ray source and the image detector.

In many cases, C-arms are connected to one end of a movable arm disposed on a base or gantry, which can be stationary or moveable. In such cases, the C-arm can often be raised and lowered, be moved from side to side, and/or be rotated about one or more axes of rotation via the moveable arm. Accordingly, such C-arms can be moved and reoriented to allow X-ray images to be taken from several different positions and angles and different portions of a patient, without requiring the patient to be frequently repositioned.

As a result, the C-arm is particularly capable of performing a cone beam computed tomography (CBCT) imaging procedure to provide real time imaging of a patient, such as during various types of interventional procedures performed on the patient. The C-shape and size or diameter of the C-arm allows a physician to access to a patient while the patient is being imaged. In order to obtain medical images of an internal structure at various angles, the C-arm may be rotated to various positions around the patient and optionally the physician to provide the necessary images of the patient.

However, the structures of the imaging system supported by the C-arm, i.e., the X-ray source, the detector, and associated cabling interconnecting the X-ray source and detector with the gantry or base, among others, requires the C-arm to be constructed to support a significant amount of weight, in excess of 250 kg. Consequently, an amount the C-arm may be rotated may limited by a structural strength of the C-arm as determined in part by the size of the C-arm and the materials used in its construction. In particular, when the C-arm is moved to position the X-ray source and detector where desired for obtaining an image of a patient, the movement creates a significant amount of inertia in the C-arm, particularly when the C-arm is moved to an imaging position at a relatively fast speed. When the C-arm is slowed and stopped, this inertia can cause vibrations in the C-arm that may irreversibly deform the C-arm if the C-arm is rotated beyond a given rotational angle for the structural strength of the C-arm.

In addition, movement produced by the radiation source while the radiation source is emitting radiation may cause the C-shaped arm to vibrate, which can be significantly accentuated at the opposed ends of the rotational range of motion for the C-arm. This vibration may cause an unwanted image artifact within a medical image acquired by the medical imaging system, thereby degrading the image quality (IQ) of the image to be used by the physician, e.g., during the interventional procedure.

To alleviate this issue, the C-arm used in these medical imaging system have been modified to be formed of materials that reduce the overall weight to be supported by the C-arm, while increasing the structural strength of the C-arm to allow this C-arm to rotate farther than C-arms made of other materials. These materials, including carbon fiber as disclosed in U.S. Pat. No. 11,864,938, entitled Imaging System With Carbon Fiber C-arm, the entirety of which is expressly incorporated herein by reference for all purposes, provide significant benefits to C-arms formed of these material. Specifically, enhancements are provided to the structural strength of the C-arm, the mechanical properties of the C-arm (i.e., stiffness), and the geometry of the C-arm which influence vibration characteristics (i.e., frequency, amplitude, and dampening) thereby reducing an amount of image artifacts within images produced by the medical imaging system with a C-arm formed at least in part of a carbon fiber material.

Nevertheless, even with the benefits provided by a C-arm constructed of alternative materials, such as carbon fiber, the C-arm must still be mounted to the gantry or base for movement with regard thereto. These mounting and support structures can be formed as rails attached along each side of the C-arm that are engaged by a carrier or trolley to hold and guide the orbital movement of the C-arm to position the radiation source and detector. Due to the high mechanical load placed on the rails, the material capable of handling these loads that is used to form the rails is steel, e.g., hardened steel, stainless steel and/or galvanized steel. However, with the length of each rail required for use with the C-arm, e.g., approximately 3-4 meters, the weight of the rails formed of steel increases the weight of the C-arm beyond the desired tolerances for the alternative materials. i.e., the carbon fiber, forming the C-arm.

Therefore, it is desirable to develop a structural mounting and support component or guide rails for moveably securing a C-arm to a gantry or base of a medical imaging system that has an increased load capacity to support the C-arm along with a reduced weight to enhance the operation of the C-arm and the medical imaging system.

### BRIEF DESCRIPTION OF THE DISCLOSURE

According to one exemplary non-limiting aspect of the disclosure, a C-shaped arm for use with a medical imaging system includes a C-shaped portion, a radiation source carried by the C-shaped portion, a radiation detector carried by the C-shaped portion, and a pair of guide rails secured to opposed sides of the C-shaped portion, wherein each of the pair of guide rails has a body formed of a lightweight material and including a pair of rod channels formed therein and a pair of rods engaged within the pair of rod channels.

According to still another aspect of one exemplary non-limiting embodiment of the disclosure, a medical imaging system includes a C-arm, a radiation source and a radiation detector carried by the C-arm, a base, a carriage coupled to the C-arm and the base, the carriage including a pair of trolleys therein, each of the pair of trolleys rotatably supporting a number of rollers thereon, wherein the C-arm has a C-shaped portion and a pair of guide rails secured to opposed sides of the C-shaped portion and engaged with the number of rollers on the pair of trolleys, wherein each of the pair of guide rails includes a body formed of a lightweight material and including a pair of rod channels formed therein and a pair of rods engaged within the pair of rod channels.

According to still another aspect of one exemplary non-limiting embodiment of the disclosure, a method of forming a C-arm for a medical imaging system includes the steps of providing a C-shaped section adapted to support a radiation source and a radiation detector at opposed ends of the C-shaped section, forming a material into a body for a guide rail, the body including a pair of rod channels disposed on opposed sides of the body, engaging a pair of rods within the pair of rod channels to form the guide rail, and securing the guide rail to a side of the C-shaped section to form the C-arm, wherein the material forming the body is selected from aluminum or steel, and wherein a diameter of each of the pair of rods is greater than a depth of each of the rod channels.

It should be understood that the brief description above is provided to introduce in simplified form a selection of concepts that are further described in the detailed description.

### BRIEF DESCRIPTION OF THE DRAWINGS

Various aspects of this disclosure may be better understood upon reading the following detailed description with reference to the drawings in which:

FIG. 1 is a side elevation view of a medical imaging system in accordance with an exemplary embodiment of the present disclosure;

FIG. 2 is a block diagram of a control mechanism of the medical imaging system of FIG. 1 in accordance with an exemplary embodiment of the present disclosure;

FIG. 3 is a is a block diagram of a computing device of the medical imaging system of FIG. 1 in accordance with an exemplary embodiment of the present disclosure;

FIG. 4 is a schematic diagram of a C-arm of the medical imaging system of FIG. 1 in a first position in accordance with an exemplary embodiment of the present disclosure;

FIG. 5 is a schematic diagram of a C-arm of the medical imaging system of FIG. 1 in a second position in accordance with an exemplary embodiment of the present disclosure;

FIG. 6 is a schematic diagram of a C-arm of the medical imaging system of FIG. 1 in a third position in accordance with an exemplary embodiment of the present disclosure.

FIG. 7 is a cross sectional view of a C-arm of the medical imaging system of FIG. 1 coupled to a guide system in accordance with an exemplary embodiment of the present disclosure.

FIG. 8 .is an isometric view of a C-arm of the medical imaging system of FIG. 1 in accordance with an exemplary embodiment of the present disclosure.

FIG. 9 is a side elevation view of the C-arm of FIG. 8 including a mounting rail thereon in accordance with an exemplary embodiment of the present disclosure.

FIG. 10 is partially broken away, cross-sectional view along line 10-10 of FIG. 9.

FIG. 11 is partially broken away, cross-sectional view along line 11-11 of FIG. 9.

FIG. 12 is partially broken away, cross-sectional view along line 12-12 of FIG. 9.

FIG. 13 is a partially broken away, sectional view along line 13-13 of FIG. 9.

FIG. 14 is a side elevational view of the mounting rail of FIG. 9 in accordance with an exemplary embodiment of the present disclosure.

FIG. 15 is a partially broken away, sectional view of the rail of FIG. 14.

FIG. 16 is a cross-sectional view along line 16-16 of FIG. 15.

FIG. 17 is a partially broken away, sectional view of a rod channel formed within the rail of FIG. 14.

FIG. 18 is a partially broken away, sectional view of a guide rod positioned within the rod channel of the rail of FIG. 14.

### DETAILED DESCRIPTION

In the following detailed description, reference is made to the accompanying drawings that form a part hereof, and in which is shown by way of illustration specific embodiments, which may be practiced. These embodiments are described in sufficient detail to enable One or more specific embodiments of the present disclosure are described below. These described embodiments are only examples of the systems and methods for a medical imaging system with a C-arm having lightweight, high load capacity guide rails. The skilled artisan will understand that specific details described in the embodiments can be modified when being placed into practice without deviating from the spirit of the present disclosure.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (i.e., a material, element, structure, number, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

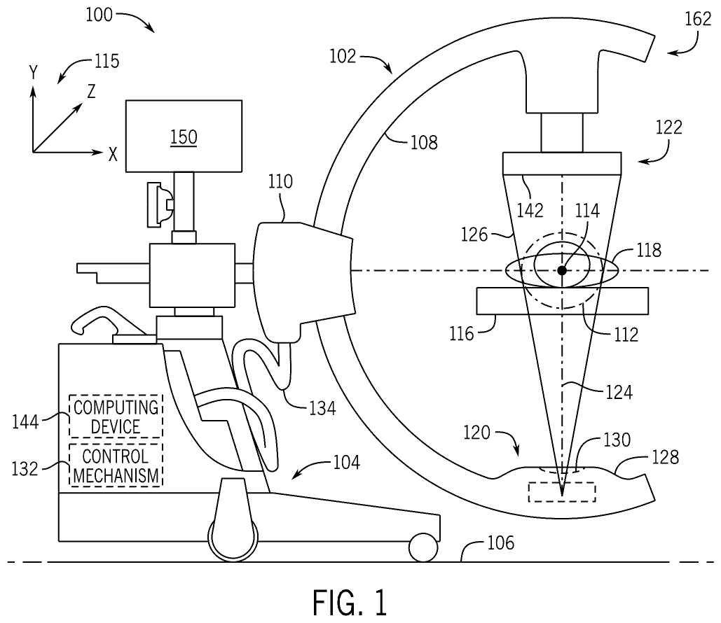

The following description relates to various embodiments for a medical imaging system with a C-arm. A medical imaging system, such as the medical imaging system shown in FIG. 1, includes a C-arm configured to rotate around at least one rotational axis. The C-arm includes a radiation source and a radiation detector at opposite ends of the C-arm.

Referring to the figures generally, the present disclosure describes systems and methods for a medical imaging system with a carbon fiber C-arm. The medical imaging system described herein (i.e., the medical imaging system depicted in FIG. 1) may be generally referred to as a radiographic medical imaging system.

Referring now to FIG. 1, a medical imaging system 100 is shown in accordance with an exemplary embodiment. The medical imaging system 100 includes a rotatable C-arm 102 that is connected to a base 104. The base 104 supports the C-arm 102 while the C-arm 102 is stationary and while rotating. The base 104 supports the C-arm 102 on a ground surface 106 on which the medical imaging system 100 sits. The C-arm 102 includes a C-shaped portion 108 that is connected to an extended portion or carriage 110. The C-shaped portion 108 may be at least partially formed of a composite material, such as carbon fiber fabric. The carbon fiber fabric may provide increased strength to the C-shaped portion 108 and/or a reduced weight of the C-shaped portion 108 relative to C-arms that include a C-shaped portion formed of a different material (i.e., steel, aluminum, etc.). The carriage 110 is rotatably coupled to the base 104 which allows the C-arm 102 to rotate about an examination region 112 and a rotational axis 114. For example, the C-arm 102 may be configured to rotate at least 180° in opposing directions relative to the base 104. As will be discussed in further detail herein, in some embodiments, the C-arm 102 may be configured to rotate at least 220°. Configuring the C-arm 102 to rotate at least 220° may provide a physician with greater access to a patient being imaged. Structural characteristics (i.e., material used to form the C-shaped portion 108) may reduce an amount of vibration and deformation the C-arm 102 experiences when the C-arm 102 rotates about the rotational axis 114. While the following describes the rotation of the C-arm 102 as rotating in the X and Y directions of the Cartesian coordinate system 115 (i.e., rotating the C-shaped portion 108 such that opposing ends of the C-shaped portion 108 are closer to or further from the carriage 110 in various positions), it is understood that the C-arm 102 may also rotate in the Z direction (i.e., rotating the C-shaped portion 108 such that opposing ends of the C-shaped portion 108 are closer to or further from a head of the patient within the examination region 112 in various positions). In addition, the base 104 can include a vertical lift column (not shown) operably connected to the carriage 110 that permits the C-arm 102 and carriage 110 to move vertically in relation to base 104. Vertical lift column can optionally include a horizontal extension arm (not shown) operably connected to the carriage 110 and the vertical lift column that permits the C-arm 102 to move perpendicularly in relation to vertical lift column by movement (e.g., horizontal movement) of the horizontal extension arm in relation to the base 104.

The medical imaging system 100 further includes a patient support 116 (i.e., couch, bed, table, etc.) that supports a patient 118 while at least a portion of the patient 118 is within the examination region 112. The medical imaging system 100 additionally includes a radiation source 120 and a radiation detector 122. The radiation source 120 and the radiation detector 122 are supported by and rotate with the C-arm 102. Furthermore, the radiation source 120 and the radiation detector 122 are positioned at opposite ends of the C-shaped portion 108 of the C-arm 102 along axis 124, where axis 124 intersects and extends radially relative to the rotational axis 114. The C-shaped portion 108 may be rotated as described above in order to adjust the position of the radiation source 120 and the radiation detector 122. Furthermore, in the embodiment depicted in FIG. 1, the position of the radiation detector 122 may be varied such that the radiation detector 122 is placed further from or closer to the radiation source 120.

During a medical imaging procedure, a portion of the patient 118 is within the examination region 112 and the radiation source 120 emits radiation 126. In one embodiment, the radiation source 120 may include an X-ray tube (not shown in FIG. 1) housed within a casing 128. The X-ray tube generates the radiation 126 which escapes the casing 128 via an outlet 130. The radiation 126 traverses the examination region 112 and is attenuated by the portion of the patient 118 that is within the examination region 112. Specifically, the radiation source 120 emits the radiation 126 towards the radiation detector 122 which is on the opposite end of the C-arm 102. The radiation source 120 emits cone-shaped radiation which is collimated to lie within an X-Y-Z plane of the Cartesian coordinate system 115 which is generally referred to as an “object plane” which is parallel to the radiation detector 122 at an isocenter of the C-arm 102.

After passing through a portion of the patient 118, the attenuated radiation is captured by the radiation detector 122. In some embodiments, the radiation detector 122 includes a plurality of detector elements (not shown) that acquire projection data. Each detector element produces an electrical signal that is a measurement of the attenuation at the detector element location. The attenuation measurements from all the detector elements in the detector 122 are acquired separately to produce a transmission profile. In one embodiment, the radiation detector 122 is fabricated in a flat panel configuration including a plurality of detector elements.

When the radiation source 120 and the radiation detector 122 are rotated with the C-arm 102 within the object plane and around the patient 118, the angle at which the radiation 126 intersects the patient 118 changes. A group of attenuation measurements (i.e., projection data) form the radiation detector 122 at one C-arm angle is referred to a “view.” A “scan” of the patient 118 includes asset of views made at different angles, or view angles, during rotation of the C-arm 102. As used herein, the term view is not limited to the use described herein with respect to projection data from one C-arm 102 angle. The term view is used to mean one data acquisition whenever there are multiple acquisitions from different angles.

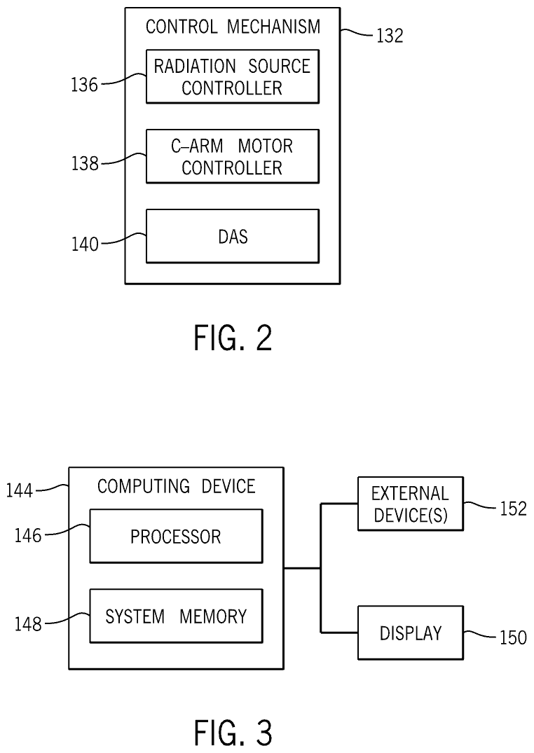

The medical imaging system 100 further includes a control mechanism 132 that is housed within the base 104. The control mechanism 132 is connected to the C-arm 102, the radiation source 120, and the radiation detector 122 via a cable 134 which allows the control mechanism to send data to/receive data from the C-arm 102, the radiation source 120, and the radiation detector 122. The control mechanism 132 controls the rotation of the C-arm 102 and the operation of the radiation source 120. Briefly turning to FIG. 2, a block diagram of the control mechanism 132 is shown in accordance with an exemplary embodiment. In one embodiment, the control mechanism 132 includes a radiation source controller 136 and a C-arm motor controller 138. The radiation source controller 136 is configured to provided power and timing signals to the radiation source 120. The C-arm motor controller 138 is configured to control a rotation speed and/or position of the C-arm 102. Furthermore, the C-arm motor controller 138 controls the rotation axis of the C-arm 102, a position of the detector 122 and thereby controlling a source to detector distance, and a location of the patient support 116. The control mechanism 132 further includes a data acquisition system (DAS) 140. The DAS 140 is configured to sample analog data received from the radiation detector 122 and convert the analog data to digital signals for subsequent processing. While FIG. 1 depicts the base 104 as including the control mechanism 132, in other embodiments the control mechanism may be separate from the base 104 (i.e., in a different room).

The C-arm 102 may be adjusted to a plurality of different positions by rotation of the C-shaped portion 108. For example, in an initial, first position shown by FIG. 1, the radiation detector 122 may be positioned vertically above the radiation source 120 relative to the surface 106 on which the medical imaging system 100 sits, with axis 124 arranged normal to the surface 106 intersecting a midpoint of the outlet 130 of the radiation source 120 and a midpoint of a detector surface 142 of the radiation detector 122. The C-arm motor controller 138 and a guide system within the extended portion 110 may adjust the C-shaped portion 108 from the first position to a different second position by rotating the C-shaped portion 108 via a coupling between the guide system and the C-shaped portion 108. In one example, the second position may be a position in which the radiation source 120 and the detector 122 are rotated 180° together relative to the first position such that the radiation source 120 is positioned vertically above the radiation detector 122, with the axis 124 intersecting the midpoint of the outlet 130 of the radiation source 120 and the midpoint of the detector surface 142 of the radiation detector 122. When adjusted to the second position, the radiation source 120 may be positioned vertically above the rotational axis 114 of the C-shaped portion 108 and the radiation detector 122 may be posited vertically below the rotational axis 114.

The medical imaging system 100 further includes a computing device 144 that is housed within the base 104. While FIG. 1 depicts the computing device 144 as housed within the base 104, in other embodiments the computing device 144 may be remote from the rest of the medical imaging system 100. As used herein, a computing device (or system) is any device/system capable of processing, storing, and/or transmitting data (i.e., tablet, handheld device, smartphone, personal computer, laptop, network computer, server, mobile communication device, etc.). The computing device 144 may be connected to a network (i.e., a wide area network (WAN), a local area network (LAN), a public network (the internet), etc.) which allows the computing device 144 to communicate with other devices on a same network. In some embodiments, the network may be regarded as a private network and may include, for example, a virtual private network.

Briefly turning to FIG. 3, a block diagram of the computing device 144 is shown in accordance with an exemplary embodiment. The computing device 144 includes a processor 146 and a system memory 148. In some embodiments, the computing device is connected to a display 150 and one or more external devices 152. The processor 146 is in communication with the system memory 148 and may execute computer readable program instructions stored in the system memory 148. As used herein, a processor may include a central processing unit (CPU), or other electronic components capable or executing computer readable program instructions (i.e., a digital signal processor, a field-programmable gate array (FPGA), a graphics processing unit (GPU), etc.). Furthermore, as used herein, a processor may include two or more of a CPU, a digital signal processor, an FPGA, and a GPU.

The system memory 148 is a computer readable storage medium. As used herein, a computer readable storage medium is any device that stores computer readable program instructions for execution by a processor and is not construed as transitory per se. Computer readable program instructions include programs, logic, data structures, modules, etc. that when executed by a processor create a means for implementing functions/acts. Computer readable program instructions when stored in a computer readable storage medium and executed by a processor direct a computer system and/or another device to function in a particular manner such that a computer readable storage medium comprises an article of manufacture. System memory as used herein includes volatile memory (i.e., random access memory (RAM) and dynamic RAM (DRAM)) and non-volatile memory (i.e., flash memory, read-only memory (ROM), magnetic computer storage devices, etc.). In some embodiments the system memory 148 may further include cache.

In one embodiment, the various methods and processes may be stored as computer readable program instructions in the system memory 148. In this embodiment, the system memory 148 includes computer readable program instructions for imaging a patient with a medical imaging system (i.e., the medical imaging system 100).

The external devices 152 include devices that allow a user to interact with/operate the computing device 144 (i.e., mouse, keyboard, touchscreen, speakers, etc.). In some embodiments, the display 150 displays a graphical user interface (GUI). The GUI includes editable fields for inputting data (i.e., patient data, imaging parameters, etc.) and further includes selectable icons. Selecting an icon and/or inputting data causes the processor 146 to execute computer readable program instructions stored in the system memory 148 which causes the processor to perform a task. For example, a user of the computing device 144 may use an external device 152 to select a “start” icon or the like which causes the processor 146 to being a medical imaging procedure.

While FIG. 1 illustrates only one computing device 144, in some embodiments, the medical imaging system 100 may include more than one computing device 144. The computing device 144 may be used for inputting or outputting imaging parameters, requesting examinations, plotting data, and/or viewing images. Furthermore, in certain embodiments, the medical imaging system 100 may be coupled to multiple displays, printers, workstations, and/or similar devices located either locally or remotely (i.e., within an institution or hospital or in a an entirely different location, etc.) via one or more configurable wired and/or wireless networks. Furthermore, in some embodiments, the base further house an internal power source (not shown) that provides electrical power to operate the medical imaging system 100. Alternatively, the base 104 may be connected to an external power source to power the medical imaging system 100. A plurality of connection cables may (i.e., cable 134) may be provided to transmit electrical power to the radiation source 120, the radiation detector 122, etc.

The computing device 144 is in communication with and provides commands to the radiation source controller 136, the C-arm motor controller 138, and the DAS 140 for controlling system operations such as data acquisition and/or data processing. In some embodiments, the computing device 144 controls operation of the radiation source controller 136, the C-arm motor controller 138, and the DAS 140 based on a user input.

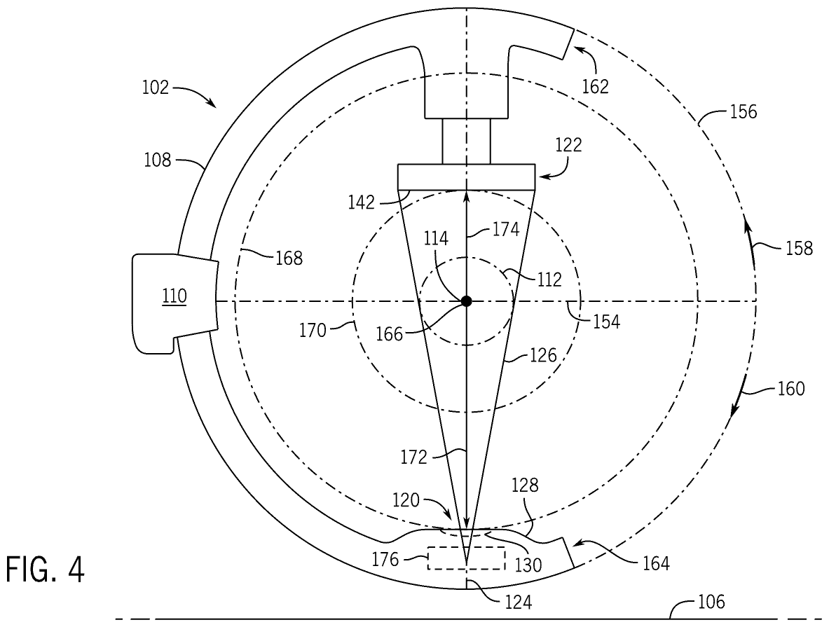

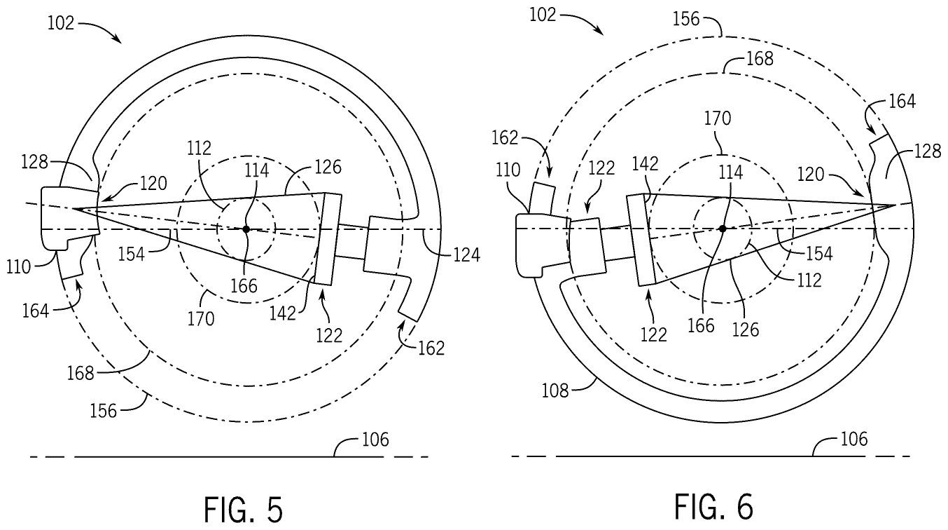

For example, the computing device 144 may send a signal to the C-arm motor controller 138 which causes the C-arm motor controller 138 to rotate the C-shaped portion 108. Referring collectively to FIGS. 4-6, various example positions of the C-arm 102 are shown. In FIGS. 4-6, the base 104 and the cable 134 have been omitted and in FIGS. 5 and 6 the outlet 130 has also been omitted for clarity.

Specifically, FIG. 4 shows the C-arm 102 in a first position in which the axis 124 between the detector surface 142 and the outlet 130 is arranged perpendicular to the ground surface 106 on which the medical imaging system 100 sits. Furthermore, as depicted in FIGS. 1 and 2, in the first position, the axis 124 is perpendicular to a horizontal axis 154. The horizontal axis 154 is parallel to the ground surface 106. FIG. 5 depicts the C-arm 102 in a second position in which the C-arm shaped portion 108 has been rotated. In the second position, the radiation source 120 is positioned closer to the carriage 110 and the radiation detector 122 is positioned further from the carriage 110 relative to the first position as shown by FIG. 4. FIG. 6 depicts the C-arm 102 in a third position in which the C-shaped portion 108 has been rotated. In the third position, the radiation source 120 is positioned further from the carriage 110 and the radiation detector 122 is positioned closer to the carriage 110 relative to the first position as shown by FIG. 4.

A rotational range of the C-shaped portion (i.e., an amount of angle through which the C-shaped portion 108 may rotate relative to the base 104) may be greater than 180° (i.e., 200°, 205°, 220°, etc.). For example, FIG. 5 may correspond to a rotation of the C-shaped portion 108 by an angle of approximately 95° around the rotational axis 114 relative to the position shown by FIG. 4, and FIG. 6 may correspond to a rotation of the C-shaped portion 108 by an angle of approximately −95° around the rotational axis 114 relative to the position shown by FIG. 4, with the C-shaped portion 108 rotating through 190° to adjust from the position shown by FIG. 6 to the position shown by FIG. 5. In each of FIGS. 2-4, the carriage 110 is maintained in position relative to the C-shaped portion 108, with the position of carriage 110 in FIGS. 4-6 being the same as the position of carriage 110 shown in FIG. 1 (i.e., with the carriage 110 not rotated relative to the ground surface 106 or horizontal axis 154).

The radiation source 120 emits the radiation 126 towards the radiation detector 122. As the C-shaped portion 108 rotates around the rotational axis 114 (i.e., while imaging the patient 118), the radiation 126 remains directed toward the radiation detector 122 due to the concurrent rotation of each of the radiation source 120 and the radiation detector 122 around the rotational axis 114. While rotating around the rotational axis 114, the C-shaped portion 108 may move along path 156 in a first direction 158 or a second direction 160. Since the radiation source 120 and the radiation detector 122 rotate around the rotational axis 114 with the C-shaped portion 108, the radiation 126 emitted by the radiation source 120 passes through the examination region 112.

While stationary or rotating, a first end 162 and a second end 164 of the C-shaped portion 108 are positioned a same distance from a center 166 of the C-shaped portion 108. The center 166 of the C-shaped portion 166 shares a same position as the rotational axis 114. For example, the C-shaped portion 108 may have a uniform radius of curvature in a direction around the center 166 (i.e., a same radius of curvature at each location along the C-shaped portion 108 in the direction around the center 166) such that the first end 162 and the second end 164 are positioned a same distance from the center 166 of the C-shaped portion 108 along the axis 124. As such, the path 156 has a same curvature and radius as the C-shaped portion 108.

As described above, the C-shaped portion 108 may rotate around the rotational axis 114. In some embodiments, C-shaped portion 108 may also rotate about horizontal axis 154. In this configuration, the C-shaped portion 108 may rotate around either of rotational axis 114 or horizontal axis 154 (or both of rotational axis 114 and horizontal axis 154), where horizontal axis 154 is orthogonal to the rotational axis 114. In the views shown by FIGS. 4-6, however, the C-shaped portion 108 is rotated only around the rotational axis 114 and not the horizontal axis 154.

Although the first end 162 and second end 164 may be positioned the same length from the center 166, each of outlet 130 and detector surface 142, may be positioned at different lengths from the center 166 since the position of the detector 122 may be varied. For example, a rotational path 168 of the outlet 130 and a rotational path 170 of the radiation detector surface 142 may be different, with each of rotational path 168 and rotational path 170 being of circular shape. The outlet 130 may move along rotational path 168 and detector surface 142 may move along rotational path 170 during conditions in which the C-shaped portion 108 is rotated around rotational axis 114 (i.e., while the patient 118 is being imaged). However, a length 172 (i.e., a diameter of the rotational path 168) may be a longer length from the center 166 to the outlet 130 than a length 174 (i.e., a diameter of the rotational path 170) from the center 166 to the detector surface 142. In one embodiment, the length 172 may be larger than the length 174 due to the radiation source 120 being seated within the C-shaped portion 108. For example, in the embodiment depicted in FIG. 4, the radiation source 120 includes an X-ray tube 176. In this embodiment, the X-ray tube 176 may be housed within casing 128 and seated within the C-shaped portion 108. For the sake of clarity, the X-ray tube 176 has been omitted in FIGS. 5 and 6.

Positioning the X-ray tube 176 within the C-shaped portion 108 may enable the outlet 130 to be positioned closer to the second end 164 compared to configurations in which an X-ray tube 176 is not seated within the C-shaped portion 108, which may result in a decreased height of the radiation source 120 as a height of the radiation source 120 may be limited by the casing 128. The resulting reduced height of the radiation source 120 may increase an amount of open space between the detector surface 142 and the outlet 130, which may enable the C-arm 102 to accommodate larger patients and/or increase ease of use of the C-arm 102. Furthermore, in some embodiments, the seated position of the radiation source 120 within the C-shaped portion 108 may increase a balance of the C-arm 102, which may reduce undesired vibration of the C-arm 102. Positioning the radiation source 120 within the C-shaped portion 102 may also increase a balance of the C-arm 102 while the C-shaped portion 108 is rotating (i.e., while the patient 118 is being imaged) and may provide a counter weight to the radiation detector 122 which may reduce a load and/or vibration of a motor of the medical imaging system 100.

Returning to FIG. 1, in one embodiment, the medical imaging system 100 includes, or is coupled to a picture archiving and communication system (PACS). In an exemplary implementation, the PACS may be further coupled to a remote system such as a radiology department information system, hospital information system, an internal and/or an external network, etc. to allow operators at different locations to supply commands, parameters, and/or gain access to image data generated by the medical imaging system 100.

The medical imaging system 100 further includes or is coupled to an image reconstructor. As previously noted, the DAS 140 samples and digitizes projection data acquired by the radiation detector 122. Subsequently, the image reconstructor uses the sampled and digitized X-ray data to perform high-speed reconstruction. In certain embodiments, the image reconstructor may form part of the computing device 144. Alternatively, the image reconstructor may be omitted and instead the computing device 144 may perform one or more functions of the image reconstructor. Moreover, the image reconstructor may be located locally or remotely and may be operatively coupled to the medical imaging system 100 via a wired or wireless network. Particularly, in one embodiment, the reconstructor may use computing resources in a “cloud” network cluster for image reconstruction.

In some embodiments the reconstructor receives projection data and the projection data undergoes preprocessing and calibration to condition the data to represent the line integrals of attenuation coefficients of the patient 118. The processed data is commonly referred to as “projections.” The acquired sets of projection data may be used for basis material decomposition (BMD). During BMD, the measured projections are converted to a set of material-density projections. The material-density projections may be reconstructed to form a pair or set of material-density maps or images of each respective basis material (i.e., bone, tissue, and/or contrast agents, etc.). The density maps or images may be, in turn, associated to form a volume rendering of the basis material in the imaged volume.

The projection data is processed to reconstruct an image that corresponds to a two-dimensional slice taken through the patient 118, or in some examples wherein the projection data includes multiple vies or scans, a three-dimensional rendering of a portion of the patient 118. Once reconstructed, a basis material image reveals internal features of the patient 118 expressed by the densities of the materials. The image may be displayed to show these features. Once displayed, a practitioner may view the image to make a medical diagnosis or to discern characteristics of a feature of interest (i.e., lesion, organ, etc.).

As used herein, the phrase “reconstructing an image” is not intended to exclude embodiments in which data representing an image is generated but a viewable image is not. Therefore, as used herein, the term image broadly refers to both viewable images and data representing a viewable image. However, some embodiments described herein generate (or are configured to generate) at least one viewable image.

In one embodiment, the reconstructor stores reconstructed images in the system memory 148. In another embodiment, the reconstructor transmits the reconstructed image(s) to the computing device 144 for generating useful patient information for diagnosis and evaluation. In certain embodiments, the computing device 144 may transmit reconstructed images and/or patient informant to the display 150. In other embodiments, the reconstructed images may be transmitted from the system memory 148 or the reconstructor to the PACS for short-term or long-term storage.

The embodiments described herein relate to the medical imaging system 100 taking the form of an X-ray imaging system (e.g., fixed X-ray imaging system) having automated C-arm motion about multiple independent (e.g., separate or different) rotational axes (e.g., 3 or more). For example, the C-arm may rotate about 3 different axes: a lateral axis, an orbital axis, and a flip-flop axis (e.g., defined by rotation about where the C-arm is coupled to an L-arm). The motion about these 3 different axes may be automated (e.g., via a motorized system including multiple motors or servomotors). The automated motion about these 3 different axes may increase the orbital range or coverage with the C-arm without having to move the patient and/or table the patient is disposed on. Motorization of all of these axes (especially the flip-flop axis) provides numerous advantages. For example, motorized motion compared to manual motion may be controlled remotely to reduce radiation exposure. In addition, motorization of all these axes enables the capture of image data for three-dimensional (3D) image generation, while still allowing the X-ray imaging system to be utilized as a general purpose C-arm imaging system. However by automating all of these axes, a general purpose C-arm imaging system may also be utilized as an accurate 3D image capturing imaging system.

Although a mobile imaging system is illustrated, the embodiments described herein may be utilized with any medical imaging system having a C-arm (e.g., a mobile C-arm imaging system). The medical imaging system 100 may utilize multiple imaging modalities (e.g., fluoroscopy, computed tomography, tomosynthesis, radiographic, magnetic resonance imaging, etc.) to acquire two-dimensional 2D and/or 3D image data. The medical imaging system 100 may be utilized for both diagnostic and interventional imaging. In addition, the medical imaging system 100 may be utilized for general purposes (e.g., general radiology, orthopedics, etc.) and special purposes (e.g., image guided surgery).

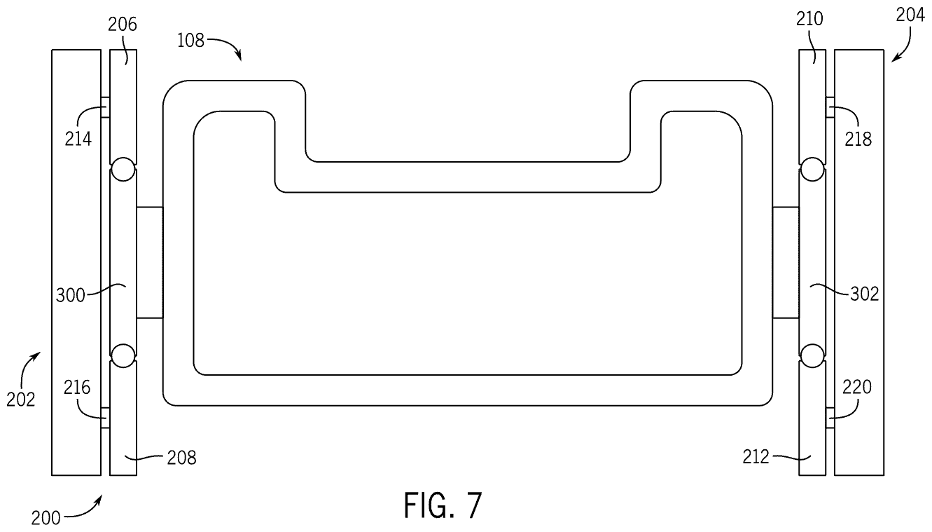

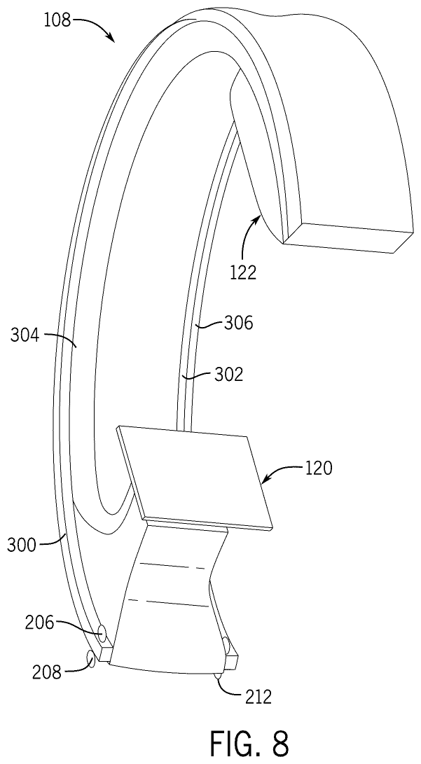

Referring now to the exemplary embodiments of FIGS. 7 and 8, the carriage 110 includes a guide system 200 coupled to the C-shaped portion 108 of the C-arm 102. As described above, the guide system 200 is operable to guide the movement of the C-arm 102 when the C-arm motor controller 138 is employed to rotate the C-arm 102 relative to the carriage 110 via a coupling between the C-shaped portion 108 and the guide system 200 formed by a first trolley 202 and a second trolley 204. In the illustrated exemplary embodiment, to guide the C-arm 102 the first trolley 202 and the second trolley 204 each engage a support and/or guide rail 300,302 secured to opposed sides of the C-shaped portion 108.

The guide system 200 further includes a first roller 206, a second roller 208, a third roller 210 and a fourth roller 212. While FIG. 7 depicts four rollers, in some embodiments, the guide system 200 may include a different number of rollers. For example, in another embodiment, each roller 206-212 may be one of two rollers that are immediately next to one another. As such, in this example, the guide system 200 includes eight rollers, as shown in FIG. 8. The first roller 206 and the second roller 208 extend from and are rotatably supported by the first trolley 202. The third roller 210 and the fourth roller 212 extend from and are rotatably supported by the second trolley 204. The first roller 206 and the second roller 208 are engaged with opposite sides of the guide rail 300, while the third roller 210 and the fourth roller 212 are engaged with opposite sides of the guide rail 302. When the C-shaped portion 108 is rotated relative to the carriage 110, the rollers 206-212 rotate about shafts 214-220 respectively (e.g., the first roller 206 rotates about the first shaft 214) as the guide rails 300,302 move relative to the carriage 110 along with the C-shaped portion 108 to guide the movement of the C-arm 102 in a given orbital rotation direction. In an exemplary embodiment for the construction of the individual rollers 206-212, the rollers 206-212 are each formed as ball bearing rollers (not shown) including number of ball bearings disposed between an inner race, which can be stationary and mounted to the C-arm 102, and an outer race, which can rotate relative to the inner race. The use of ball bearing rollers for rollers 206-212 on both sides of the C-arm 102 significantly enhances trajectory repeatability for the movement of the C-arm 102, thereby enhancing image quality, by reducing the amount of lateral play present in the rollers 206-212, as opposed to using needle roller bearings on one or both sides of the C-arm 102.

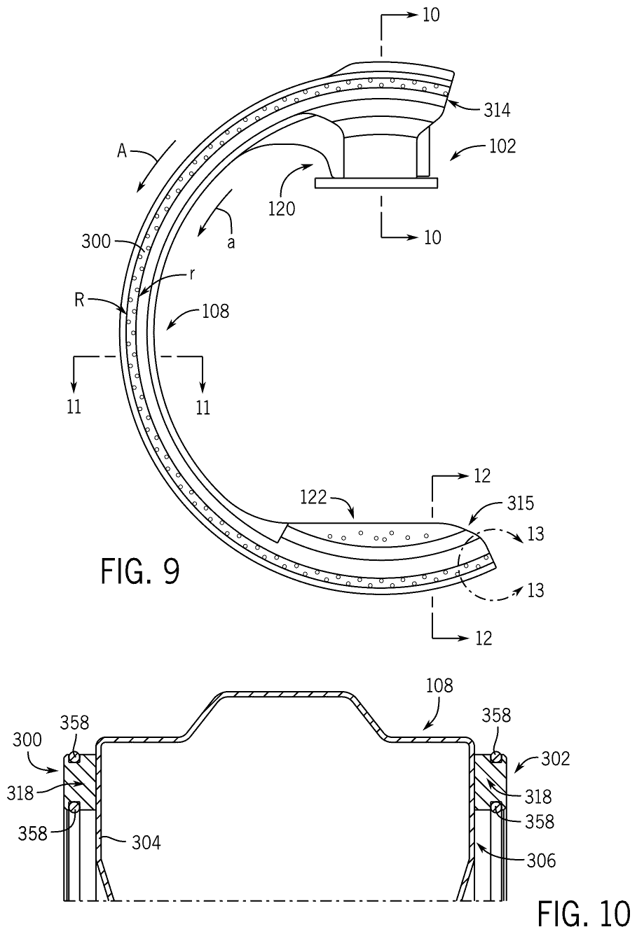

Referring to the illustrated exemplary embodiments of FIGS. 9-13, the C-shaped portion 108 of the C-arm 102 is illustrated having the guide rails 300,302 attached thereto. The guide rails 300,302 are secured to opposed sides 304,306 of the C-shaped section 108 in any suitable manner, such as by a number of fasteners 308 engaged inserted through openings 310 in the guide rails 300,302 and engaged within bores 312 disposed in the C-shaped portion 108. The guide rails 300,302 extend along the perimeter of the C-shaped portion 108 from a position adjacent an X-ray source end 314 of the C-shaped portion 108 to a position adjacent a detector end 315 of the C-shaped portion 108. At each end, the rail 300,302 is engaged with a stop 316 mounted to the C-shaped portion 108. The stop 316 covers the associated end of the guide rail 300,302 and functions to limit the travel of the rollers 206-212 along the guide rails 300,302, thereby preventing the rollers 206-212 from becoming disengaged from the guide rails 300,302.

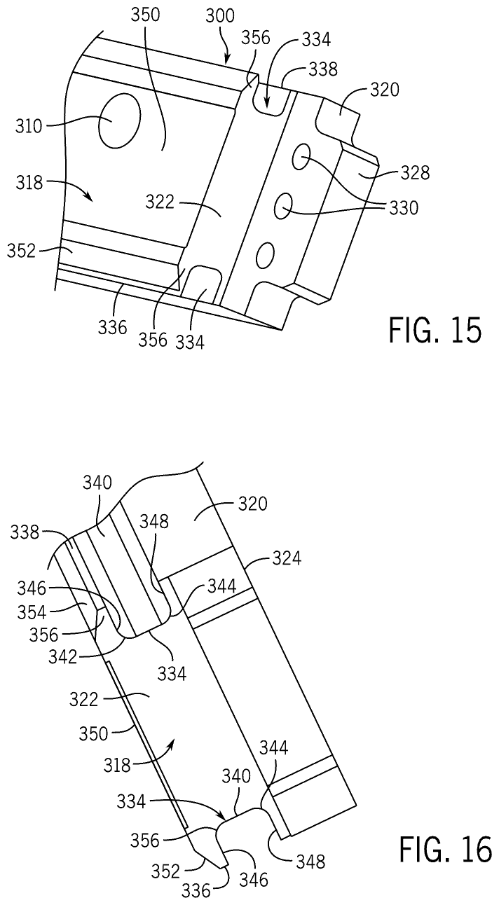

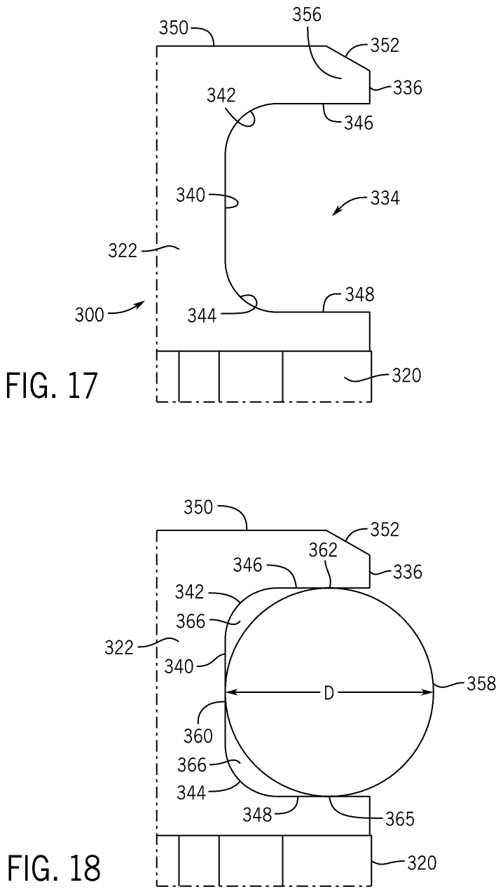

Looking now at the exemplary embodiments illustrated in FIGS. 14-17, the rails 300,302 each include a body 318 in which the openings 310 are formed. The body 318 is formed of a suitable lightweight material, such as aluminum or also steel. The body 318 is formed as a single or unitary piece of material, which can be shaped into the body 318 of the guide rail 300,302 in a suitable manufacturing process. In a particular embodiment, to negate the need for machining the body 318 into the desired configuration for the guide rail 300,302, the material forming the body 318 can be used in an extrusion molding process to form the material into the body 318. The extrusion of the material forming the body 318 provides high level accuracy in the shape of the body 318, and can eliminate the need for further processing steps on the body 318 that are required after machining. Additionally, forming the body 318 as a single piece also negates the potential for play along the guide rail 300, 302 as no pieces or fasteners are employed to secure parts of the rails 300,302 to one another on the C-shaped section 108.

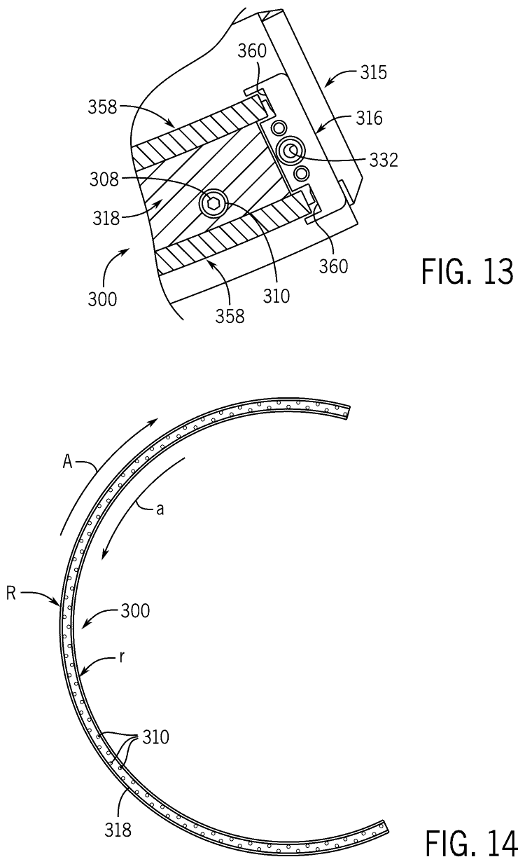

The body 318 is formed with a generally rectangular cross-section, including an inner section 320 and an outer section 322. The inner section 320 is disposed on the C-shaped portion 108 and includes a flat inner surface 324 placed against the corresponding side 304,306 of the C-shaped portion 108. The inner section 320 functions to space the outer section 322 from the side 304,306 of the C-shaped portion 108 on which the guide rail 300,302 is secured to enable the roller 206-212 to move freely with regard to the guide rail 300,302 without contacting the C-shaped portion 108. Further, at each end of the inner section 320 the body 318 includes a flange 328 that extends axially outwardly from the inner section 320. The flanges 328 include apertures 330 therein that are adapted to receive fasteners 332 therethrough that are used to secure the body 318 to the C-shaped section 108, as well as to secure the stop 316 to the guide rail 300,302 and to the C-shaped portion 108 (FIG. 13).

The outer section 322 is also formed with a generally rectangular cross-sectional shape, and is disposed directly on the inner section 320 opposite the flat inner surface 324. In addition, the outer section 322 includes a pair of rod channels 334 disposed in opposed sides 336,338 of the outer section 322 and extending the entire length of the body 318. The side 336 is disposed along an inner arc a of the body 318 and has a shorter overall length than the side 338 disposed along the outer arc A of the body 318. The rod channels 334 in each side 336,338 are each formed with a generally U-shaped cross-section, including a flat central surface 340, a pair of curved corners 342,344 located at each end of the central surface 340, and a pair of flat side surfaces 346,348 extending between the rounded corners 342,344 and the adjacent side 336,338 of the outer section 322. Opposite the inner section 320, the outer section 322 also defines a flat, outer surface 350. The outer surface 350 terminates with a pair of tapered ends 352,354 extending from the outer surface 350 to the adjacent side 336,338. The tapered ends 352,354 each define a thin, wedge-shaped compression section 356 between the tapered ends 352,354 and the adjacent rod channel 334.

Looking now at FIGS. 10-13 and 18, to provide the surfaces on the guide rails 300,302 for engagement by the rollers 206-212, a pair of rods 358 are disposed within the rod channels 334. The rods 358 each have lengths slightly longer than the rod channels 334 in the body 318, such that the ends of the rods 358 are positioned within aligned recesses 360 located in the stops 316 secured to the flanges 328. In the illustrated exemplary embodiment of FIGS. 10-13, the rods 358 are each formed as monolithic, unitary structures with a circular cross-section, though other configurations for the cross-section of the rods 358 are also contemplated as being within the scope of the present invention. The diameter D of the rods 358 is greater than the depth of the rod channel 334 from the side 336,338 of the outer section 322 to the central surface 340, such that a portion of the rod 358 protrudes beyond the side 336,338 when the rod 358 is disposed within the rod channel 334. The rods 358 are formed of a high tensile strength material, such as a metal, and more particularly hardened steel. This enables the rods 358 to provide a durable and high load bearing surface on the guide rails 300,302 which the rollers 206-212 can repeatedly travel along during the orbital movement of the C-arm 102.

To form the guide rails 300,302, in a first embodiment initially the material forming the guide rail 300,302 is extruded into the shape for the body 318, including the rod channels 334 and the tapered ends 352,354 of the outer surface 350. The body 318 is extruded as a straight component without any curvature. Subsequent to the extrusion, the body 318 is bent in a suitable manner to form the body 318 with a curvature corresponding to the shape of the C-shaped section 108 with the inner radius r and the outer radius R.

Concurrently, or subsequently to the formation and bending of the body 318, the rod 358 is formed in a suitable process, such as extrusion, and then bent in a suitable manner to form the rod 358 with a curvature corresponding to the shape of the body 318 and the C-shaped section 108. Additionally, the rod 358 is formed to have a diameter slightly greater than the width of the rod channel 334 between the side surfaces 346,348. The length of the rod 358 will correspond to the length of the rod channel 334 along the inner arc a or outer arc A in which the rod 358 is to be placed.

To assemble the rod 358 within the body 318, the rod 358 is press fit or shrink fit into the corresponding rod channel 334. In this process, as best shown in FIG. 18, the larger diameter rod 358 is pressed into the rod channel 334 with sufficient force to contact the rod 358 against the central surface 340. When fully inserted within the rod channel 334, the circular cross-section of the rod 358 has a limited number of points or areas of contact 360,362,364 with the body 318 within the rod channel 334. As a result of this, the upper limit of the tolerance range for the diameter D of the rods 358 is increased since the limited points/areas of contact 360,362,364 allow for spaces 366 to be defined between the rod 358 and the rod channel 334 that are not in contact with one another. The process to form the interference fit (e.g., the press fit or shrink fit) between the rod 358 and the rod channel 334 can address the placement of rods 358 with diameters D at or near the upper range of the tolerances for the rods 358 without affecting the engagement of the rod 358 in the rod channel 334 or the integrity of the guide rail 300,302.

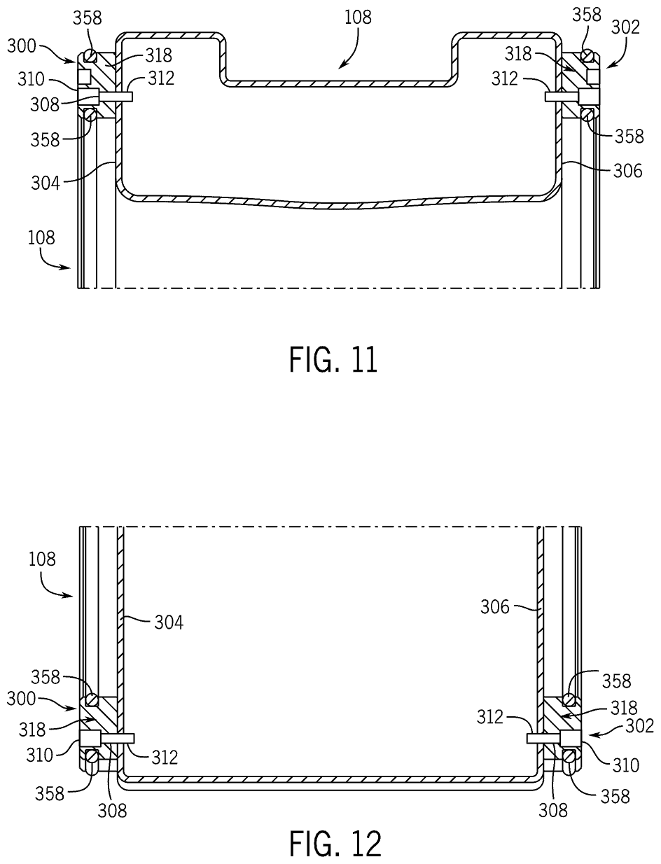

Once the rods 358 are positioned within each of the rod channels 334, the guide rail 300,302 can be secured to the associated side 304,306 (FIGS. 11 and 12) of the C-shaped portion 108 using fasteners 308 and the C-arm 102 can be engaged with the carriage 110 on the medical imaging device 10.

In an alternative method for manufacturing the guide rails 300,302, the body 318 can be formed with a straight configuration in an extrusion process, similarly to that described previously. After the extrusion of the body 318, the rods 358 can be press fit or shrink fit into the rod channels 334 formed within the straight body 318 to form the structure for the guide rails 300,302. Subsequently to the engagement of the rods 358 within the rod channels 334, the guide rails 300,302 can be bent in a suitable manner to provide the guide rails 300,302 with the curvature corresponding to the associated side 304,306 of the C-shaped portion 108. The curved guide rail 300,302 can then be secured to the associated side 304,306 (FIGS. 11 and 12) of the C-shaped portion 108 and the C-arm 102 can be engaged with the carriage 110 on the medical imaging device 10.

In an alternative embodiment to the above methods, after or during the engagement of the rod 358 within the associated rod channel 334 in either method, the wedge-shaped compression section 356 can be compressed against the rod 358. This compression provides an easier/less complex manufacturing process for the guide rail 300,302, as strict tolerances for the rod channels 354 are not required, produces less material fatigue within the body 318 and allows for precise positioning to the rod 358 within the rod channel 334 on the guide rail 300,302 in conjunction with the depth of the rod channel 334.

In another alternative embodiment for the above-described methods, after formation of the body 318 for the guide rail 300,302, by extrusion or machining, the body 318 can be secured to the C-shaped section 108 prior to the insertion of the rods 358. After attachment of the body 318 to the C-shaped section 108, the rods 358 can be engaged, e.g., press fit, shrink fit, etc., into the rod channels 334, with the optional compression of the wedge-shaped sections 356 against the rods 358.

With the above described methods of manufacture and corresponding structure(s) for the guide rails 300,302, the curved guide rails 300,302 provide a lightweight no-play structure along which the C-arm 102 (which can also be formed of a lightweight structural material, e.g., carbon fiber) mounted to the guide rails 300,302 can move within the orbital direction in an accurate and precise manner with minimal vibration to enhance the image quality for the images obtained by the medical imaging device 10 including the C-arm 102.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A C-shaped arm for use with a medical imaging system comprising:
a C-shaped portion;
a radiation source carried by the C-shaped portion;
a radiation detector carried by the C-shaped portion; and
a pair of guide rails secured to opposed sides of the C-shaped portion, wherein each of the pair of guide rails comprises:
a body formed of a lightweight material and including a pair of rod channels formed therein; and
a pair of rods engaged within the pair of rod channels.

2. The C-shaped arm of claim 1, wherein the body is formed as a unitary structure.

3. The C-shaped portion of claim 1, wherein each of the pair of rods is formed as a unitary structure.

4. The C-shaped arm of claim 1, wherein the body is formed of aluminum, and each of the pair of rods are formed of hardened steel.

5. The C-shaped arm of claim 1, wherein a diameter of each of the pair of rods is greater than a depth of each of the rod channels.

6. The C-shaped arm of claim 1, wherein each of the pair of rod channels defines three areas of contact between the rod channel and rod engaged therein.

7. The C-shaped arm of claim 1, wherein each of the pair of rod channels includes a compression section along one side of each of the pair of rod channels.

8. A medical imaging system comprising:
a C-arm;
a radiation source and a radiation detector carried by the C-arm;
a base;
a carriage coupled to the C-arm and the base, the carriage including a pair of trolleys therein, each of the pair of trolleys rotatably supporting a number of rollers thereon,
wherein the C-arm comprises:
a C-shaped portion; and
a pair of guide rails secured to opposed sides of the C-shaped portion and engaged with the number of rollers on the pair of trolleys, wherein each of the pair of guide rails comprises:
a body formed of a lightweight material and including a pair of rod channels formed therein; and
a pair of rods engaged within the pair of rod channels.

9. The medical imaging system of claim 8, wherein the body is formed as a unitary structure.

10. The medical imaging system of claim 8, wherein each of the pair of rods is formed as a unitary structure.

11. The medical imaging system of claim 8, wherein the body is formed of aluminum, and each of the pair of rods are formed of hardened steel.

12. The medical imaging system of claim 8, wherein a diameter of each of the pair of rods is greater than a depth of each of the rod channels.

13. The medical imaging system of claim 8, wherein each of the pair of rod channels defines three areas of contact between the rod channel and rod engaged therein.

14. A method of forming a C-arm for a medical imaging system, the method comprising the steps of:
providing a C-shaped section adapted to support a radiation source and a radiation detector at opposed ends of the C-shaped section;
forming a material into a body for a guide rail, the body including a pair of rod channels disposed on opposed sides of the body;
engaging a pair of rods at least partially within the pair of rod channels to form the guide rail; and
securing the guide rail to a side of the C-shaped section to form the C-arm,
wherein the material forming the body is selected from aluminum or steel, and
wherein a diameter of each of the pair of rods is greater than a depth of each of the rod channels.

15. The method of claim 14, wherein the step of forming the body comprises extruding the material to form the body as a unitary structure.

16. The method of claim 14, further comprising the step of bending the body into a curved shape after forming the body.

17. The method of claim 14, further comprising the step of bending the body into a curved shape after engaging the pair of rods within the pair of rod channels.

18. The method of claim 14, wherein the step of engaging the pair of rods at least partially within the pair of rod channels comprises forming an interference fit between the pair of rods and the pair of guide channels.

19. The method of claim 18, wherein the step of forming the interference fit comprises press fitting or shrink fitting the pair of rods within the pair of rod channels.

20. The method of claim 18, wherein the step of forming the body comprises forming a compression section adjacent one side of each of the pair of rod channels on the body, and wherein the step of forming the interference fit between the pair of rods and the pair of rod channels further comprises compressing the compression sections against the pair of rods.
